(19) United States
(12) Patent Application Publication
(10) Pub. No.: US 2023/0381402 A1
(43) Pub. Date: Nov. 30, 2023

(54) WASHING DEVICE FOR THE EAR, NOSE AND THROAT AND THE LIKE

(71) Applicant: TECOS S.A.S. DI ALESSANDRO TODESCO E C., Filago (BG) (IT)

(72) Inventor: Alessandro Todesco, FILAGO (BG) (IT)

(21) Appl. No.: 18/032,826

(22) PCT Filed: Oct. 18, 2021
(86) PCT No.: PCT/EP2021/078763

§ 371 Date: Apr. 20, 2023

(30) Foreign Application Priority Data
Oct. 20, 2020 (IT) 102020000024712

(51) Int. Cl. A61M 3/02

(52) U.S. Cl. CPC A61M 3/0245; A61M 3/0202; A61M 2205/366; A61M 2205/505; A61M 2210/0618; A61M 2210/065; A61M 2210/0662

(57) ABSTRACT

A washing device for the ear, nose and throat and the like (10) for domestic use or in medical offices which comprises a body (12) within which the components functional to the operation of the device are housed, a container element (14) defining a tank for a saline solution, a cannula (16) for use by the individual for the washing operations, a cable (18) for connecting to the power supply network.

The present invention relates to a washing device for the ear, nose and throat and the like.

More in particular, the present invention relates to a device for otolaryngological washing treatments and, in particular but not exclusively, for washing the nasal cavities in domestic contexts or in medical offices.

As is known, the nasal washing treatment has the function, in both adults and children, of decongesting the nasal cavities and allowing to restore proper breathing as well as filtering activities of the nasal cavities themselves and this because the nasal washing treatments allow to neutralize, drain and “discharge” viruses, bacteria and other substances which are harmful and polluting to the nasal cavities.

Therefore, nasal washing treatments are useful for the treatment and prevention of diseases such as colds (by virtue of the fact that they remove excess mucus or “plugs”), sinusitis and rhinitis and, in general, keep the upper respiratory tract free and efficient.

Excess mucus or “plugs” can be removed by blowing the nose, with the expulsion of the mucus into a tissue.

However, tissues tend to irritate the nose and are often bothersome to skin and, moreover, often cause and favour an inversion of phlegm, directing it into the ear tubes when the individual, in the act of blowing his nose, presses the nostrils creating a pressure contrary to the direction which the mucus should follow to exit.

Such a situation results in the mucus staying in the tubes for months, with the consequence that for the removal thereof, treatments with nasal insufflation with sulphur-containing solutions carried out at thermal stations or centres or other specialized facilities are necessary, with the consequent related costs.

The above insufflation treatments are typically performed by skilled personnel.

Other insufflation treatments can also be carried out by the individual person (or by a doctor) using saline liquid (isotonic, hypertonic or sulphuric solutions or preparations containing thermal water or sea water or even simple water and salt) which must pass through the nasal cavities (with the liquid which is introduced from one nostril to exit from the other nostril and be discharged together with any impurities).

In addition, said saline liquid must be introduced at a temperature such as to allow the dissolving of the nasal mucus without causing damage to the nasal mucous membranes; the suitable temperature for such treatments is typically in a range from 35° to 38° C.

However, the products/devices traditionally used for nasal washings comprise saline solutions used at ambient temperature, i.e., at temperatures between 10° and 20° C. and, therefore, ineffective for the nasal washing action in accordance with what has been described above.

Other known devices such as, for example, the device described in WO2020/097560, refer to the use of fluids at temperatures between 44 and 52° C. functional to the treatment of bleeding in body cavities; such temperature ranges, although functional to haemostatic functions, are not suitable for nasal washing functions as they are not comfortable for the patient undergoing treatment.

Furthermore, the traditional washing products/devices include the use of non-pressurized solutions which, therefore, struggle to “mechanically” drag the mucus out of the nostril opposite that of the introduction of the washing solution.

The object of the present invention is to overcome the drawbacks described above.

More in particular, it is an object of the present invention to provide a washing device for treatments for the ear, nose and throat and the like adapted to allow washing at favourable temperatures for dissolving mucus and, more in particular, at temperatures between 35° and 38° C.

It is a further object of the present invention to provide a device for washing treatments which does not involve problems of reversing the phlegm with the directing thereof towards the ear tubes.

It is a further object of the present invention to provide a device for washing treatments adapted to allow pressurized washes to be carried out so as to allow a mechanical dragging of mucus and impurities in the direction of the nostril opposite that of the introduction of the washing liquid/solution.

It is a further object of the present invention to provide a washing device which can also be used in domestic contexts without the need to resort to specialized medical personnel.

A further object of the present invention is to provide users with a washing device for treatments for the ear, nose and throat and the like adapted to ensure high strength and reliability over time and such that it can be easily and economically created.

These and other objects are achieved by the invention having the features according to claim 1.

According to the invention, a washing device for the ear, nose and throat and the like is provided for domestic use or in medical offices which comprises a body within which the components functional to the operation of the device are housed, a container element defining a tank for a saline solution, a cannula for use by the individual for the washing operations, a cable for connecting to the power supply network.

Advantageous embodiments of the invention appear from the dependent claims.

The constructive and functional features of the washing device for the ear, nose and throat and the like of the present invention can be better understood from the following detailed description, in which reference is made to the attached drawings which represent a preferred and non-limiting embodiment, in which:

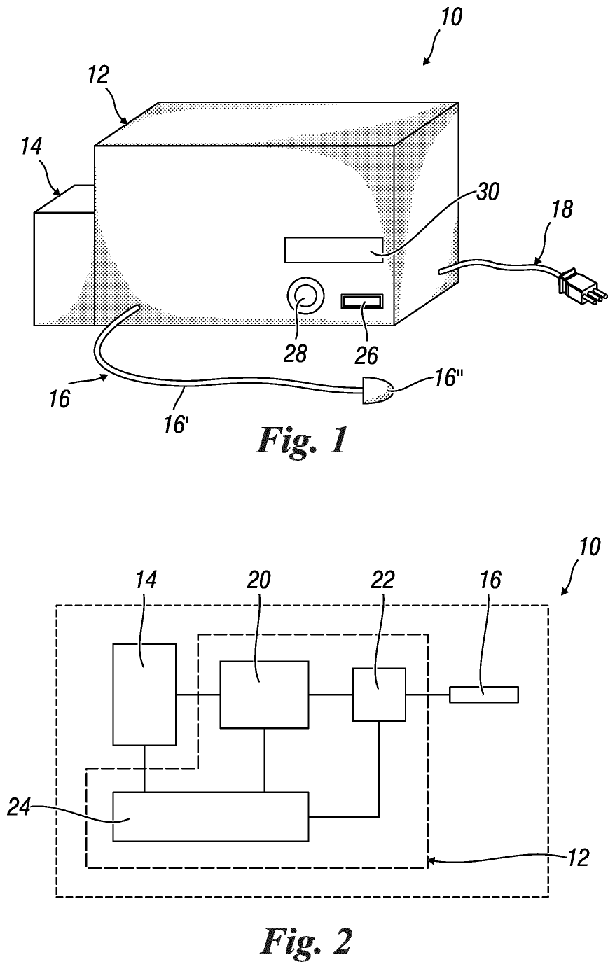

FIG. 1 schematically illustrates the washing device for treatments for the ear, nose and throat and the like according to a preferred embodiment of the invention;

FIG. 2 schematically illustrates a block diagram of the functional components of the washing device of the invention.

With reference to the aforementioned figures, the washing device for treatments for the ear, nose and throat and the like, indicated overall with 10 in the figures, comprises a body 12 defined by a substantially box-like element within which the components functional to the operation of the device are housed, a container element 14 defining a tank for a saline solution, a cannula 16 for use by the individual for the washing operations as better explained in the following and a cable 18 for connecting to the power supply network.

The body 12 houses a pump 20 which withdraws the saline solution from the container element 14 defining the tank and sends it pressurized to a heat exchanger 22 in which, by means of an electronic probe (not shown in the figures) driven by an electronic control 24, the saline solution is heated to the required temperature with a tolerance of ±1° C.

The same body 12 externally comprises an on/off switch 26 of the device, a selector 28 for adjusting the temperature of the saline solution and a possible display 30 or, alternatively, LED (Light Emitting Diode) type light indicators with the function of displaying device operation information such as, for example, set temperature value, desired temperature reached, saline solution level in the container element 14 defining the tank and the like.

According to an alternative embodiment, the selector 28 is of the digital type and is integrated into a touch (or swipe) type display.

The container element 14 defining a tank has, as previously described, the function of containing the saline solution (at a temperature between 10° and 20° C.) and can be arranged outside the body 12 or integrated therein (in this case the body 12 may comprise a door to allow filling said container element 14 defining the tank).

The heat exchanger 22 is of a known type (and is therefore not the subject of detailed description herein) and, in the preferred embodiment of the figures, is of the type comprising a stainless steel plate on which a ceramic layer is deposited on which an electrical circuit is printed (made of a high temperature copper alloy—for example copper-beryllium) defining the heating element and above which a layer of glass is melted to define a sealing and electrically insulating surface above which a stainless steel plate provided with a spiral channel is welded within which the saline solution withdrawn from the tank and fed by means of the pump flows, heating.

Said heat exchanger may also be of the induction heating block type or of the thick-film surface heater type.

The electronic control 24 receives, by means of the probe, a signal related to the temperature value of the input saline solution entering the exchanger and provides a consent signal for heating said solution up to a temperature value set by the user by means of the selector 28 and keeps the saline solution at the set temperature value (the temperature of the pressurized saline solution transited in the heat exchanger is between 30° and 38° C.).

The cannula 16 for the individual's use is functional to allow the flow of the pressurized and heated saline solution exiting the heat exchanger 22 into the individual's nasal cavity or nostril.

For this purpose, said cannula 16 comprises a tube 16′ connected to the body 12 and, in particular, to the heat exchanger 22 and a cap or tip 16″ stabilized to the tube 16′ on the opposite side with respect to that connecting to the body 12, with said cap preferably rounded or “ogival” in shape, functional to an easy introduction into the individual's nasal cavity.

In an alternative embodiment, the device of the invention may comprise an accessory element defined by a lance or tube having the function of allowing a flow of steam functional to a cleaning of the cannula 16 carrying the saline solution.

The device of the invention, described above with particular reference to a use for nasal washings, can also be used for ear washings (external ear washing) or eye washings simply by replacing the cannula 16 or the cap or tip 16″ thereof with a cap or tip having a shape suitable for the specific washing operation (ear or eye washing) and may also comprise an additional cannula or tube connected to the cap or tip to allow the exit of the liquid during the washing.

As it can be seen from the foregoing, the advantages achieved by the washing device for the ear, nose and throat and the like of the present invention are evident.

The washing device for treatments of the ear, nose and throat and the like advantageously allows washing at favourable temperatures for dissolving mucus and, more in particular, at temperatures between 35° and 38° C., with said temperatures being adjustable by the user with a tolerance of ±1° C.

A further advantage is that the device of the invention uses a pressurized saline solution which allows an easy and rapid expulsion of the mucus.

Further advantageous is the fact that the washing device for treatments of the ear, nose and throat and the like of the present invention does not involve problems of reversing phlegm with the directing thereof towards the ear tubes.

Further advantageous is the fact that the device of the invention is simple and easy to use and can also be used in domestic contexts without the need to resort to specialized medical personnel.

Further advantageous is the fact that the device of the invention can be used, as an alternative to nasal washing, also for an eye or ear washing and, in the latter case, the temperature of the saline solution between 35° and 38° C. favours the removal of wax from the ears without the need to use tools such as cotton swabs which are ineffective, dangerous to use as well as harmful to the environment.

Although the invention has been described above with particular reference to an embodiment, given merely by way of non-limiting example, numerous modifications and variations will be apparent to a person skilled in the art in the light of the above description. Therefore, the present invention intends to embrace all the modifications and variations which fall within the scope of the following claims.

1. A washing device for the ear, nose and throat and the like (10) for use in a domestic context or in medical offices, characterized in that it comprises a body (12) within which the components functional to the operation of the device are housed, a container element (14) defining a tank for a saline solution, a cannula (16) for use by the individual for the washing operations, a cable (18) for connecting to the power supply network, the pump (20) adapted to withdraw a saline solution from the container element (14) and to send it pressurized to a heater means of the saline solution processed by the pump (20) at a temperature between 35° and 38° C. with a tolerance of ±1° C., the body (12) comprises an electronic control means (24) of said pump (20) and of said heating means.

2. The washing device according to claim 1, characterized in that the heating means comprise a heat exchanger (22) and an electronic probe driven by the electronic control means (24).

3. The washing device according to claim 1, characterized in that the body (12) externally comprises a switch (26) for switching the device on/off, a selector (28) for adjusting the temperature of the saline solution with a tolerance of 1° C. and means for indicating the operation of the device.

4. The washing device according to claim 3, characterized in that the indicator means of the operation of the device comprise a display (30).

5. The washing device according to claim 3, characterized in that a selector (28) is of the digital type integrated in the touch-type display.

6. The washing device according to claim 1, characterized in that the cannula (16), functional to allow a flow of the saline solution inside the otolaryngological cavity of the individual, comprises a tube (16′) connected to the heat exchanger (22) of the body (12) and a cap or tip (16″) stabilized to the tube (16′) on the opposite part with respect to that connecting to the body (12) functional to an introduction into the otolaryngological cavity of the individual.

7. The washing device according to claim 6, characterized in that the cap or tip (16″) is rounded or “ogival” in shape, functional to an easy introduction inside the nasal cavity of the individual.

8. The washing device according to claim 1, characterized in that it comprises an accessory element defined by a nozzle or tube for a flow of steam functional to a cleaning of the cannula (16).

9. The washing device according to claim 1, characterized in that the container element (14) defining a tank for a saline solution is arranged outside the body (12).

10. The washing device according to claim 1, characterized in that the container element (14) defining a tank for a saline solution is integrated with the body (12).
